(19) United States
(12) Patent Application Publication
(10) Pub. No.: US 2015/0042958 A1
(43) Pub. Date: Feb. 12, 2015

(54) AERIAL PROJECTION DISPLAY WITH DUAL REFLECTORS

(71) Applicant: Wavien, Inc., Valencia, CA (US)

(72) Inventor: Kenneth LI, Castaic, CA (US)

(21) Appl. No.: 14/453,001

(22) Filed: Aug. 6, 2014

Related U.S. Application Data
(60) Provisional application No. 62/019,653, filed on Jul. 1, 2014.
(60) Provisional application No. 62/011,706, filed on Jun. 13, 2014.
(60) Provisional application No. 61/977,849, filed on Apr. 10, 2014.
(60) Provisional application No. 61/976,063, filed on Apr. 7, 2014.
(60) Provisional application No. 61/895,485, filed on Oct. 25, 2013.
(60) Provisional application No. 61/862,694, filed on Aug. 6, 2013.

(51) Int. Cl. G03B 21/28; A61B 3/032; A61B 3/00; A61B 3/028; G02B 27/22; G03B 31/00

(52) U.S. Cl. CPC G03B 21/28; G02B 27/22; G03B 31/00; A61B 3/0041; A61B 3/0285; A61B 3/032
USPC 351/246; 353/7

(57) ABSTRACT

An aerial projection display project an image of an object to a remote location for viewing. The display includes a pair of spherical, parabolic, or elliptical reflectors positioned and oriented relative to one another such that, when the object is placed at one or more predetermined locations, an image of such object is transmitted to the first reflector of said pair, reflected by said first reflector to the second reflector, and refocused by the second reflector as an undistorted 3-D image. In a second embodiment, the reflectors are paraboloid reflectors sharing a common axis of revolution. In another embodiment, the reflectors are elliptical reflectors each having a pair of foci. The foci lie along a common axis, and one of said foci is common to both reflectors. In a third embodiment, the reflectors are spherical reflectors The first reflector reflects a distorted image of the object to the second reflector, which corrects the distorted image from said first reflector to project a substantially undistorted image of the object.

### CROSS-REFERENCE TO RELATED APPLICATIONS

The present application claims priority on the following U.S. provisional patent applications:

No. 62/019,653, filed on Jul. 1, 2014;

No. 62/011,706, filed on Jun. 13, 2014;

No. 61/977,849, filed on Apr. 10, 2014;

No. 61/976,063, filed on Apr. 7, 2014;

No. 61/895,485, filed on Oct. 25, 2013; and

No. 61/862,694, filed on Aug. 6, 2013.

### BACKGROUND OF THE INVENTION

Aerial projection display of objects or images has become increasingly used in advertisements and promotions of products in various circumstances. Such displays are usually done using a kiosk. The actual product or image (hereafter referred to collectively as the “object”) is contained within the kiosk. An image of the object is projected to another location, for example in front of the kiosk, so that the object appears to be floating in the air to a person standing in front of the kiosk.

More particularly, the object is placed inside the kiosk and an imaging system creates a 3D image of the object, either inside the kiosk or in front of, the kiosk, at a position close to the viewer. The 3D image which is visible to the viewer appears to be real and can be touched. Such systems typically use a plurality of optical elements such as mirrors, beam splitters, optical filters, and polarizers to project the image.

Examples of aerial projection systems are disclosed in U.S. Pat. No. 6,817,716, U.S. Pat. No. 6,808,268, U.S. Pat. No. 6,568,818, U.S. Pat. No. 5,311,357, U.S. Pat. No. 5,552,934, U.S. Pat. No. 5,311,357, and U.S. Pat. No. 4,802,750.

FIG. 1 illustrates one type of known aerial projection system which projects an image using a concave spherical mirror 16. The object 10 is placed adjacent the optical axis 12 of the concave mirror. A flat, partially reflective mirror 14 is placed in the optical axis 12 and oriented to redirect the image of the object 10 onto the concave mirror 16. The reflected image from the concave mirror is directed outwardly along the optical axis 12 and passes through the partially reflective mirror 14 to be focused as a floating image 18 a distance in front of the concave mirror 16.

The partially reflective mirror typically transmits approximately 50 percent of the light from the object and reflects the remaining 50 percent. Because light rays from the object need to pass through the partially reflective mirror 14 twice, there is a substantial loss of brightness. With a reflective mirror that reflects 50 percent of the incoming light rays, the maximum output of the viewable image is only 25 percent of the original brightness, making the system very inefficient. Moreover, as indicated by arrow 19, a portion of the light reflected back by the concave mirror 16 is reflected by the partially reflective mirror 14 back onto the object, creating a ghost image. The ghost image lowers the contrast of the light coming from the object and produces other undesirable effects, requiring the use of a wave plate to mitigate the problem.

In FIG. 1, the flat mirror is used in order to project the beams of light from the object onto the spherical mirror along the optical axis 12 of the mirror 16. Such system can be referred to as an “on axis” system. FIG. 2, in contrast, shows an off-axis optical system where the object and the projected image are on opposite sides of the optical axis. There is no need to employ a partially reflective mirror 14, thereby improving the transmission efficiency and eliminating the ghost image. However, the system shown in FIG. 2 is undesirable in that it distorts the image depending upon the angle between the object and the optical axis.

### SUMMARY OF THE INVENTION

An aerial projection display project an image of an object to a remote location for viewing. The display includes a pair of spherical, parabolic, or elliptical reflectors positioned and oriented relative to one another such that, when the object is placed at one or more predetermined locations, an image of such object is transmitted to the first reflector of said pair, reflected by said first reflector to the second reflector, and refocused by the second reflector as an undistorted 3-D image. In a second embodiment, the reflectors are paraboloid reflectors sharing a common axis of revolution. In another embodiment, the reflectors are elliptical reflectors each having a pair of foci. The foci lie along a common axis, and one of said foci is common to both reflectors. In a third embodiment, the reflectors are spherical reflectors The first reflector reflects a distorted image of the object to the second reflector, which corrects the distorted image from said first reflector to project a substantially undistorted image of the object.

### BRIEF DESCRIPTION OF THE DRAWINGS

FIG. 1 is a schematic, side view of a known type of aerial projection display system;

FIG. 2 is a schematic, side view of another known type of aerial projection display system;

FIGS. 3-7 are schematic, side views of examples of aerial projection display systems according to the invention employing dual paraboloid reflectors;

FIGS. 8-10 are schematic, side views examples of aerial projection display systems according to the invention employing a pair of ellipsoid reflectors;

FIG. 11 is a schematic, side view of a prior art aerial projection display system employing a spherical reflector;

FIG. 12 is a schematic, side view of an aerial projection display system according to the invention which employs a pair of spherical reflectors;

FIGS. 13(a) and 13(b) are schematic side views of a spherical reflector and a corresponding lens model, respectively;

FIGS. 14(a) and 14(b) are schematic drawings of perpendicular and parallel plane optical paths illustrated using the lens model of FIG. 13(b);

FIGS. 15(a) and 15(b) are schematic drawings of another example of perpendicular and parallel optical paths illustrated using the lens model of FIG. 13(b);

FIGS. 16-17 are schematic drawings of other configurations of a pair of spherical reflectors according to the invention;

FIGS. 18-20 are schematic drawings of embodiments of the invention which employ a pair of paraboloid reflectors for projecting an image;

FIG. 21 is a schematic drawing of a panoramic floating image display using dual paraboloid reflectors;

FIG. 22 is a schematic drawing of a panoramic floating image display using dual elliptical reflectors;

FIG. 23 is a schematic drawing of a 4-D image display system using a pair of angled paraboloid reflectors;

FIG. 24 is a graph showing the relative positions of an object and its image when using the display system of FIG. 23;

FIG. 25 is a schematic drawing of the 4-D image display system of FIG. 23 when used to project a moving display inside of a building to form an image on the sidewalk outside the building;

FIG. 26 is a schematic drawing of the 4-D image display system of FIG. 23 when used as part of a communication system between persons in two different room;

FIG. 27 is a schematic drawing of the 4-D image display system of FIG. 23, in which the object is a LCD panel;

FIG. 28 is a schematic drawing of a wide angle floating image system using a pair of paraboloid reflectors;

FIG. 29 is a schematic drawing showing the extent of the reflectors of FIG. 28 around the axis of rotation;

FIG. 30 is a schematic drawing of another embodiment of a wide angle floating image system using a pair of paraboloid reflectors;

FIG. 31 is a schematic drawing of another embodiment of a wide angle floating image system using a pair of paraboloid reflectors;

FIG. 32 is a schematic drawing of another embodiment of a wide angle floating image system using a pair of paraboloid reflectors;

FIG. 33 is a schematic drawing of another embodiment of a wide angle floating image system using a pair of paraboloid reflectors;

FIG. 34 is a schematic drawing showing the extent of the reflectors of FIG. 28 around the axis of rotation;

FIG. 35 is a schematic drawing of another embodiment of a wide angle floating image system using a pair of paraboloid reflectors with an additional stage;

FIG. 36 is a schematic drawing of a typical equipment setup for determining an eyeglass prescription for a patient needing glasses;

FIG. 37 is a schematic drawing of a 4-D system according to the invention for determining a prescription for eyeglasses;

FIG. 38 is a schematic drawing of an alternative system according to the invention for determining an eyeglass prescription; and

FIG. 39 is a schematic drawing of another system according to the invention for determining an eyeglass prescription.

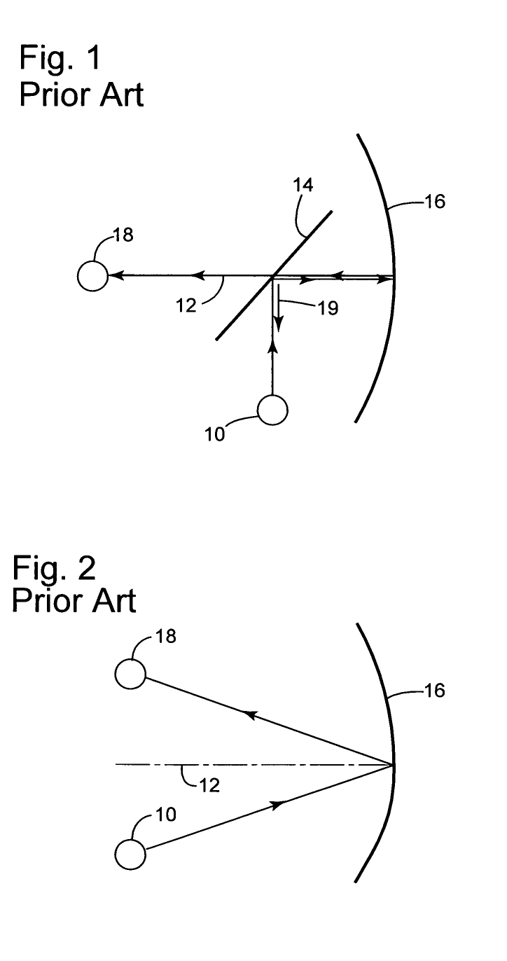

### DETAILED DESCRIPTION OF THE INVENTION

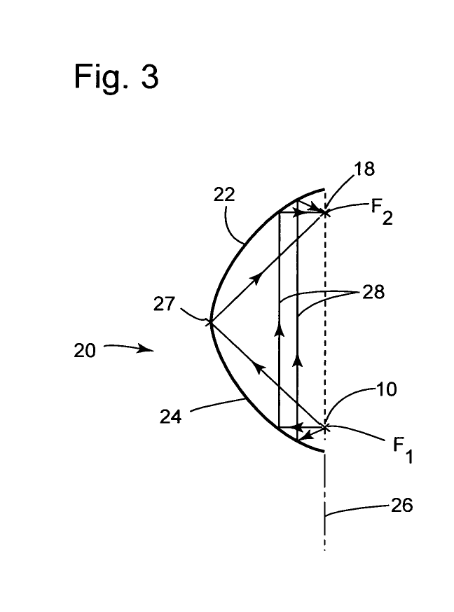

FIG. 3 illustrates a first embodiment of an aerial projection display 20 according to the invention. The display 20 includes a pair of symmetrical paraboloid reflectors 22, 24, which represent a portion of a parabolic surface generated by revolution around an axis of revolution. As used herein, a pair of paraboloid reflectors means that the two parabolas from which the paraboloid reflectors are formed face one another.

In the case of FIG. 3, the two paraboloid reflectors are symmetrical and share a common axis of revolution 26. The portion of the paraboloid to be used to form each reflector 22, 24 is determined by the desired viewing angle. A smaller portion provides a smaller viewing angle. If a 180 degree section is used, the resulting view of the image of the projected object becomes panoramic. The vertical angle used also depends on the desired viewing angle.

The two paraboloid reflectors 22, 24 meet along a common plane 27 (which is perpendicular to the surface of the drawing) and have focuses F1 and F2, respectively. Light emitted from an object 10 placed at the focus F1 will reflect off the paraboloid reflector 24 and be collimated to extend in a direction which is parallel to the common axis 26. Such collimated light 28 then reflects off of the second paraboloid reflector 22 and the image is refocused at the focus F2. Thus, the object 10 will appear as a 3-D floating image at focus F2. Because each light ray from the object 10 is refocused at target F2 in a symmetric fashion, the system has unit magnification. In other words, the image 18 of the object 10 created at focus F2 will have the same size as the object 10 at focus F1. As used herein, the term “object” can refer to a physical object or to an electronic display.

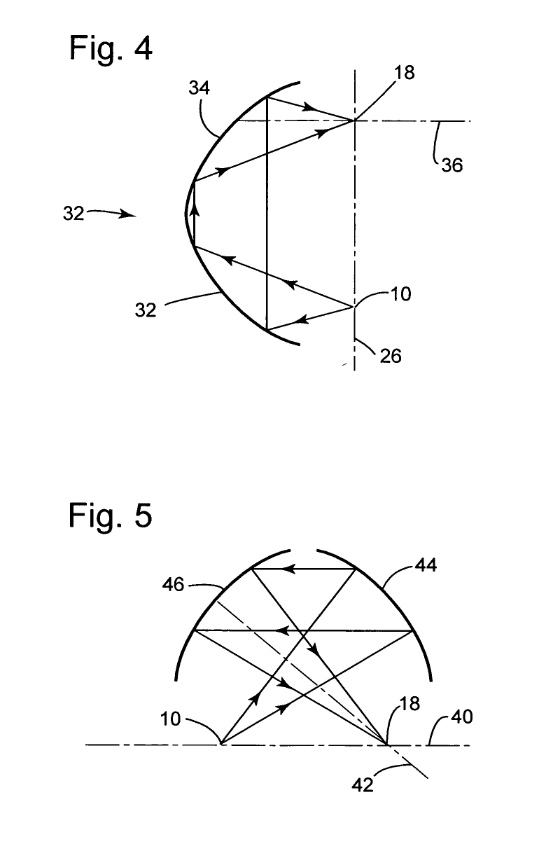

FIG. 4 shows an alternative embodiment of an aerial projection display 32 which uses a smaller section of the paraboloids than FIG. 3, providing a smaller viewing angle. Light from the object 10 is collimated by the first paraboloid reflector 32a, incident on the surface of the second paraboloid reflector 34, and refocused to form the final image 18. In the case of FIG. 4, the object 10 is positioned at a point on the common axis of revolution 26 such that the viewing axis 36 of the user is perpendicular to the axis of revolution 26. As also shown in FIG. 4, the axis of revolution 26 of the two paraboloid reflectors 32, 34 is moved forward, away from the reflector surfaces, so that the image 18 is likewise projected at a distance away from the reflectors 32, 34.

FIG. 5 shows an embodiment using a pair of paraboloid reflectors 44, 46 where the viewing axis 42 is not perpendicular to the axis of revolution 40. The object 10 is positioned on the axis of rotation 40 at the focal point of the far paraboloid reflector 44, so that light from the object 10 is incident only the far paraboloid reflector 44. Light rays from the object 10 are collimated by the paraboloid reflector 44 and directed toward the reflector 42, which refocuses the image 18 at the focal point of the reflector 42. In the FIG. 5, embodiment, the viewing axis 42 and axis of rotation 40 cross one another, allowing more flexibility in the design of the kiosk system.

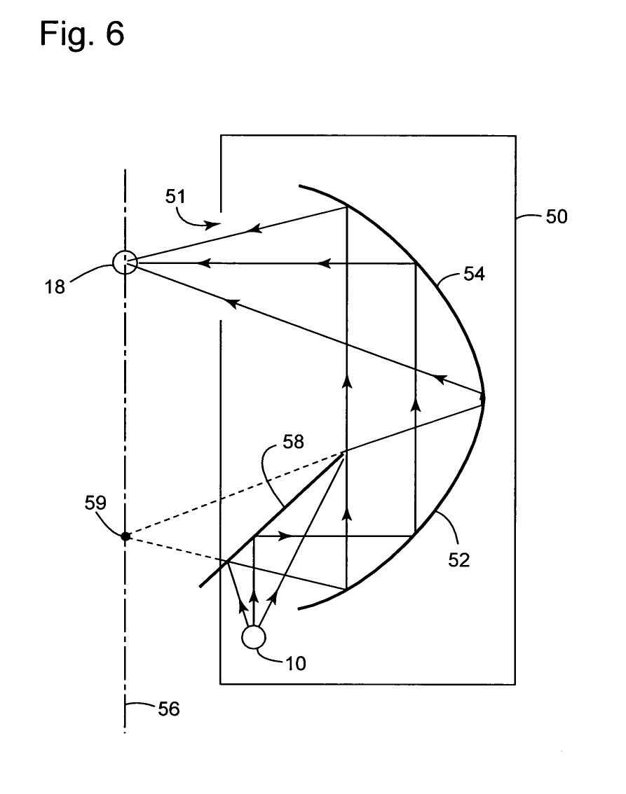

FIG. 6 schematically shows a kiosk 50 as part of an aerial display system using a pair of symmetrical paraboloid reflectors 52, 54. The parabolic axis of revolution 56, which is common for both reflectors, is located outside of the kiosk 50 so that the image 18 of an object 10 located inside the kiosk 50 is transmitted through an opening 51 in the kiosk 50 and appears in front of the kiosk. The object 10 is placed near the bottom of the kiosk 50. A planar mirror 58 reflects light rays from the object 10, turning the light path 90 degrees, thereby fainting a virtual object 59 outside of the kiosk. The mirror 58 is angled relative to the object 10 so that light rays reflected by the mirror impact the first reflector 52. Such light rays are reflected by the first reflector 52 and collimated to extend toward the second reflector 54 in a direction parallel to the axis of revolution 56. The second reflector 54 then refocuses the image 18 at the focal point of the second reflector 54, which is at a point along the axis of revolution 56.

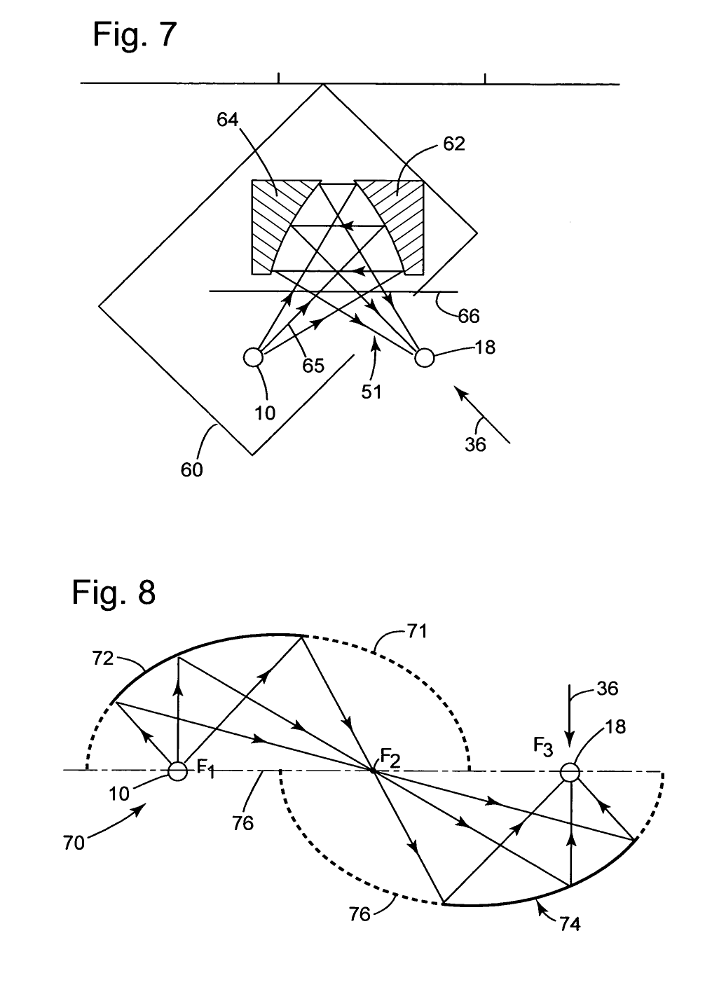

FIG. 7 schematically shows another kiosk 60 for creating an aerial display image 18 of an object 10 outside of the kiosk 60 using a pair of paraboloid reflectors 62, 64 configured similar to FIG. 5. In FIG. 7, the object axis 65 (i.e., the axis from the object to the center of the first reflector 62) and viewing axis 36 are perpendicular to one another. The reflectors 62, 64 are angled such that their common parabolic axis of revolution 66 is at a 45 degree angle relative to the object axis 65 and the viewing axis 36. Such configuration allows the kiosk 60 to be placed on a shelf or table top (not shown).

The foregoing examples employ two paraboloid reflector segments that have the same shape. As a result, the image formed is the same size as the object. In order to generate images which are either magnified or smaller than the object, reflector segments may be used which are not the same as one another.

FIG. 8 shows an alternative embodiment of an aerial display system 70. The system 70 employs a pair of ellipsoid reflectors 72, 74, each constituting a portion of an ellipsoid 71, 71a. Each ellipsoid faces a common ellipsoid axis 76 and is positioned on opposite sides of such axis 76. The first ellipsoid reflector 72 has a focus at F1 and at F2. The second ellipsoid reflector 74 has a focus at F2 and F3. The object 10 is placed at the focus F1. Light originating from the object 10 is reflected by the first ellipsoid reflector 72 through focus F2 onto the reflective surface of the second ellipsoid reflector 74, and refocused at focus F3. The image passing through focus F2 is a distorted image of the object 10. Reflecting the distorted image off the second reflector 74 imparts an equal and opposite distortion to the image coming from focus F2, so that the image at focus F3 will be an undistorted image.

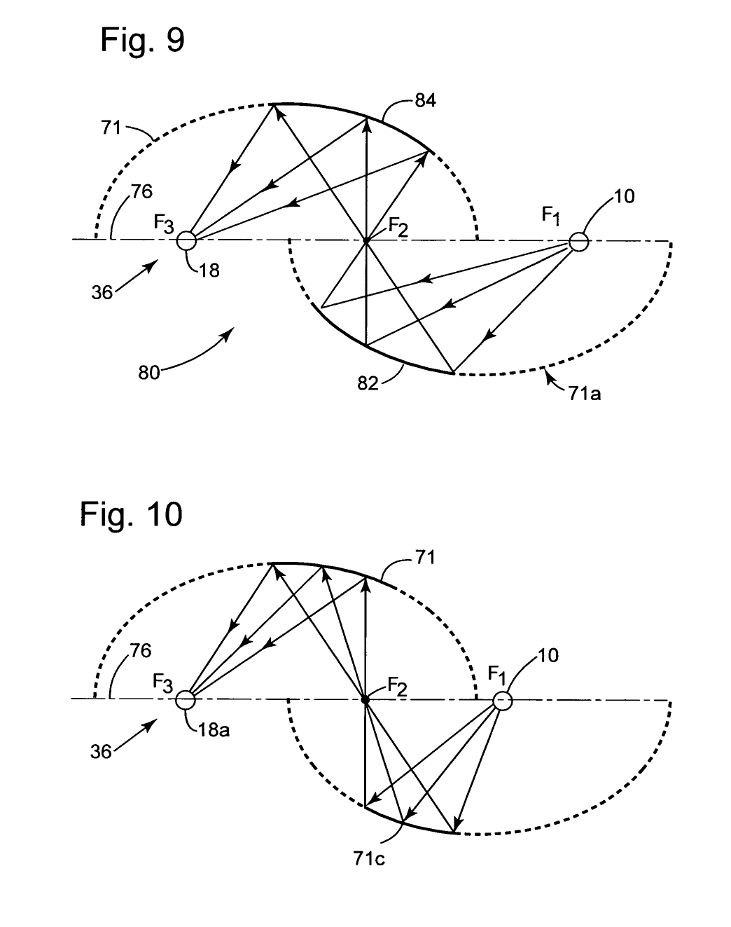

FIG. 9 shows an aerial display system 80 which is a modification of the aerial display system 70 of FIG. 8, also employing a pair of ellipsoid reflectors 82, 84. The FIG. 9 embodiment uses a different portion of the ellipsoid 71, 71a that FIG. 8. The two reflectors 82, 84 use complementary reflector portions of the ellipsoids, 71, 71a.

FIG. 10 shows another modification of the display system of FIG. 8 or 9, in which ellipsoids 71, 71c of different sizes are employed. Again, the two ellipsoids 71, 71c share a common ellipsoid axis 76 and have a common focus F2. It is preferable that the ellipticities are substantially the same. In the case of FIG. 10, the first ellipsoid 71c with the focuses F1 and F2 is smaller than ellipsoid 71 with focuses F2 and F3, so that the image 18a is magnified relative to the object 10. The opposite may be done to produce a reduced size image (in other words, the object 10 placed at focus F3 of FIG. 10 will be projected as a smaller image 18 at F1).

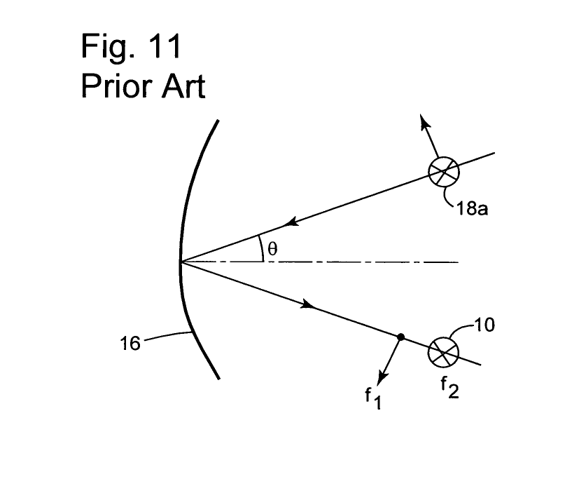

FIG. 11 shows a prior art aerial display with a spherical reflector 16 similar to FIG. 2. The reflector 16 is used for an off-axis configuration with the reflector 16 tilted in a plane with an angle θ. Stated differently, relative to the optical axis of rotation 40 of the spherical reflector 16, the object 10 is placed off-axis at an angle θ. For small angles, the effective focal length of the spherical reflector 16 will be different between the plane parallel to the optical axis 30 and the plane perpendicular to the axis of rotation 40. The difference can be expressed as follows:

f1(parallel)=f cos(θ)

f2(perpendicular)=f/f cos(θ)

where f is the original on-axis focal length.

As a result of such difference, the image 18a of an object 10 formed by an off-axis reflector will be distorted due to the difference in focal lengths in two different planes. In order to overcome such distortion, the invention employs a second spherical reflector configured such that it is tilted by the same amount as angle θ, but with the axis of rotation of the second spherical reflector perpendicular to the axis of rotation of the first spherical reflector. As a result, the plane with a shorter focal length of the spherical reflector will be combined with the longer focal length plane of the second spherical reflector, thereby compensating for the effect of the focal length changes due to tilting. In other words, the second spherical reflector is positioned and angled to distort the image received from the first spherical reflector by an equal but opposite amount, thereby producing an undistorted image.

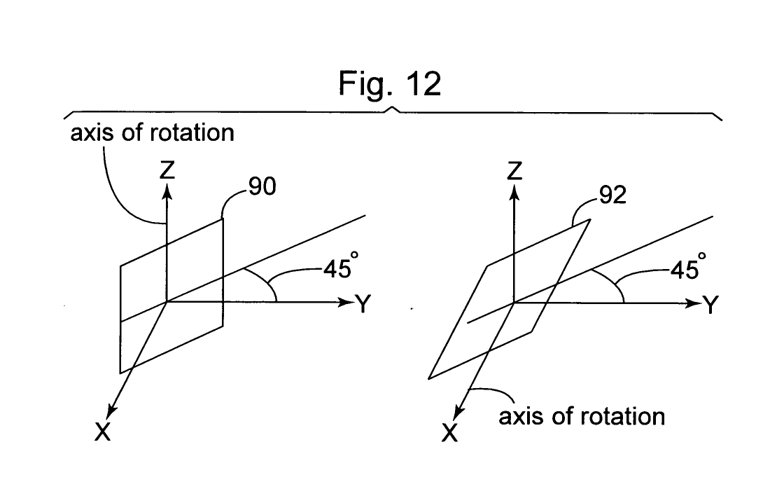

FIG. 12 shows an example of such embodiment. For simplicity in illustrating the orientation of the two spherical reflectors, the spherical reflectors 90, 92 are shown as squares. In the xyz reference system, the z-axis is the axis of rotation of the first reflector 90. The z-axis extends through the first spherical reflector 90. The first spherical reflector 90 is rotated by 45 degrees relative to the x and y-axes. In the same xyz coordinate system the x-axis for the second spherical reflector 92 is the axis of rotation. The x-axis passes through the second spherical reflector 92. The second spherical reflector 92 is rotated 45 degrees relative to the y and z-axes. In such a manner, light from an object which travels along the x-axis toward the first spherical reflector 90 is reflected by an angle of 90 degrees along the y-axis towards the second spherical reflector 92. The second spherical reflector then reflects such light along the z-axis. By selecting proper spacing and focal lengths, objects placed along the x-axis of the first spherical reflector will form a real image along the z-axis of the second spherical reflector with reduced distortion compared to a single spherical reflector system.

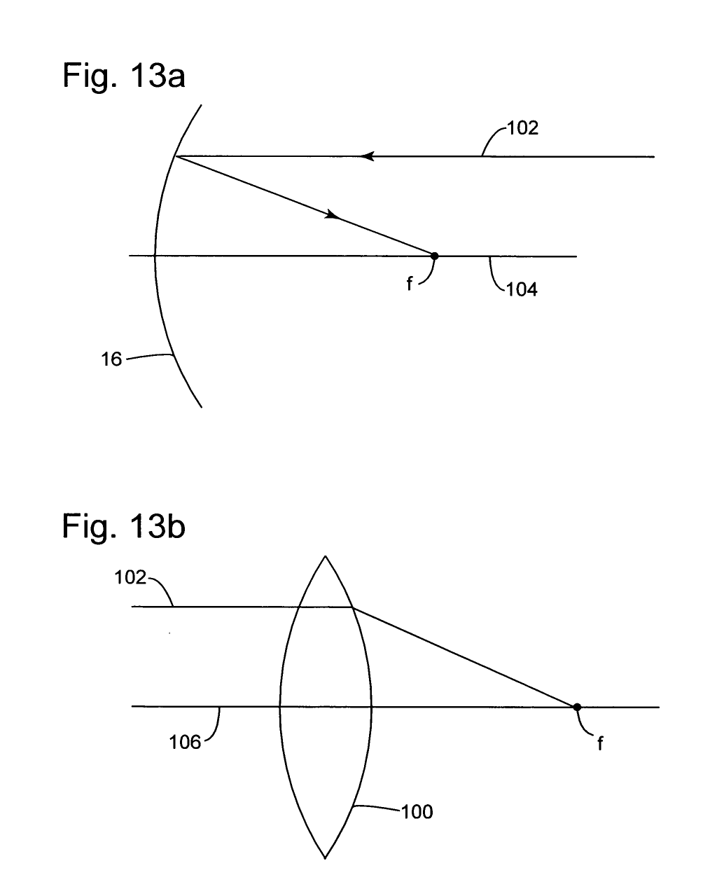

FIGS. 13a and 13b schematically show an alternate way to illustrate a spherical reflector 16. FIG. 13a shows the reflector 16 itself. FIG. 13b shows the reflector as a lens model with a lens 100. In each case, light rays 102 which are parallel to the axes 104, 106 of the reflector 16 and lens 100, respectively, are focused on the focal point f.

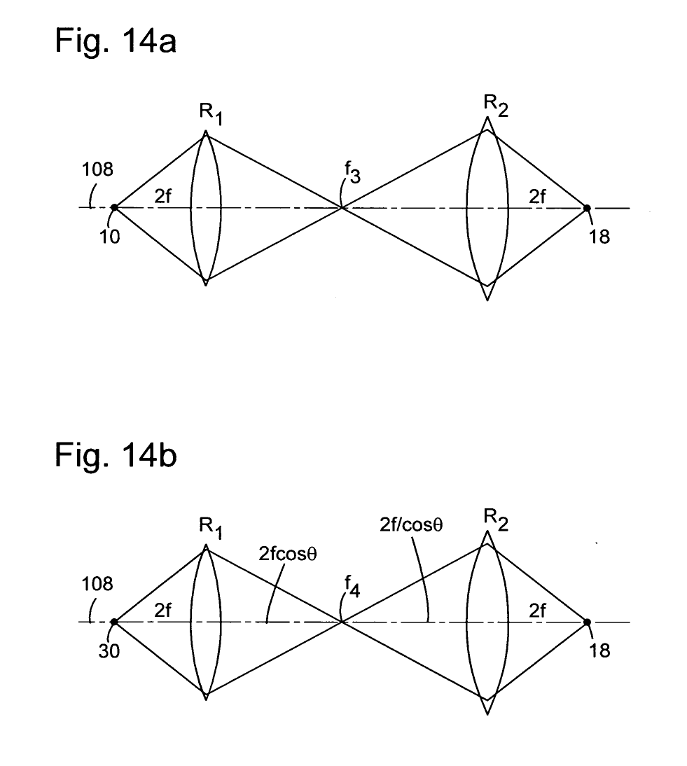

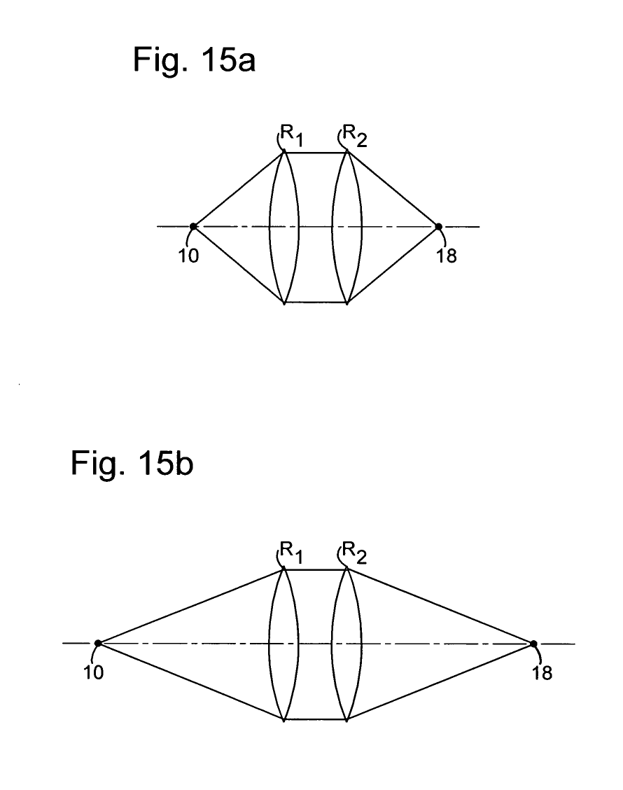

FIGS. 14a and 14b illustrate an embodiment of the invention using the lens model of FIG. 13b. FIG. 14a schematically shows a perpendicular side view of two spherical reflectors, represented by lens R1 and R2, which are perpendicular to the optical axis 108. FIG. 14a shows the effective focal lengths in a plane perpendicular to the off-axis angle, and FIG. 14b shows the effective focal lengths in a plane parallel to such angle.

In both FIGS. 14a and 14b, the two lenses R1 and R2 are separated by a distance of 4f, where “f” represents the focal length of the lenses. Note that FIG. 13 shows focusing parallel beams at “f,” whereas FIGS. 14a and 14b show imaging which is at 2f. When an object 10 is placed along the axis 108 at a distance of approximately 2f from lens R1, the lens R1 refocuses the image at f3 in FIG. 14a and at f4 in FIG. 14b. The locations of f3 and f4 relative to the lenses R1 and R2 are not the same.

In the case of FIG. 14a, the distance from the reflector R1 to the focal point 13 is 2f/cos θ, whereas the distance from focal point 13 is 2f cos θ. For the parallel configuration of FIG. 14b, the opposite is the case—the distance from R1 to focal point f4 is 2f cos θ, and the distance from the focal point f4 to the second lens R2 is 2f/cos θ. In both cases, however, the total distance between lenses R1 and R2 is 2f (1/cos θ+cos θ), which is approximately equal to (within less than one percent error of) 2f for angle ranges θ (see FIG. 11) from 0 to over 30 degrees. Thus, the system using two spherical reflectors, which are positioned and angled as described in connection with FIG. 12, a image may be produced with very little distortion

FIGS. 15a and 15b show an alternate configuration in which the spherical reflectors R1 and R2, again represented by lenses, are placed in close proximity to one another (less than 2f). In the case of the perpendicular analysis, depicted in FIG. 15a, the two lenses R1 and R2 have focal lengths f/cos θ and f cos θ, respectively and a combined focal length of f/cos θ+f cos θ. In the parallel analysis, shown in FIG. 15b, the lens R1 will have a focal length off cos θ and the lens R2 will have a focal length of f/cos θ. Again the two lenses R1 and R2 will have a combined focal length of f/cos θ and f cos θ. As a result, the distortion of the image created by lens R1, both in the plane parallel to the off-axis angle θ and in the plane perpendicular to the off-axis angle θ, will be completely compensated for when the image passes through the second lens R2.

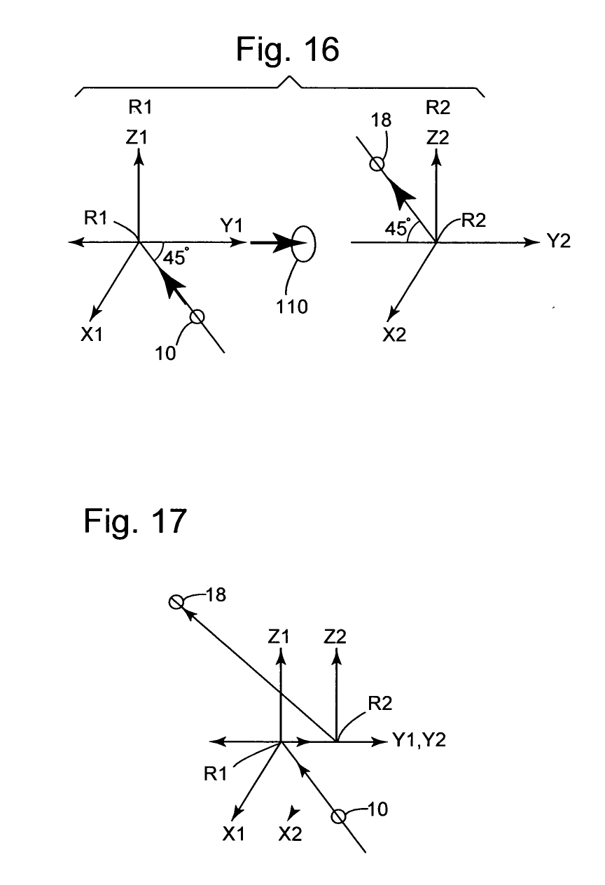

FIG. 16 is a schematic drawing illustrating the intermediate images between the two spherical reflectors R1 and R2. The object 10 is placed in the x-y plane at a 45 degree angle thereto, and is reflected by the first spherical reflector R1 along the y-axis. The intermediate image 110 is directed to the second reflector R2, which is angled to reflect the reflected image in a direction which is in the x-z plane in a direction which is angled at 45 degrees between the x and z-axes. Thus, the second reflector R2 will be at a 45 degree angle which is equal to, and the opposite of, the 45 degree angle of the first reflector R1. By configuring the reflectors R1, R2 in such a manner, substantially all distortion present in the intermediate image 110 is compensated for by the counter-distortion introduced by reflector R2.

FIG. 17 schematically shows another configuration in which the spherical reflectors R1 and R2 are placed close to one another to form a single equivalent reflector without forming an intermediate image.

The embodiments of FIGS. 11-17 all employ a pair of spherical reflectors or equivalent lenses. Depending on the size of the object, the desired viewing angle, and dimensions of the reflectors, the tilt angle can be adjusted to minimize or completely eliminate any distortion of the projected image.

Spherical reflectors can be made at a lower cost than paraboloid reflectors. Thus, the embodiments of FIGS. 11-17 is preferred when the mechanical constraints of, and specifications for, the system allow for such usage.

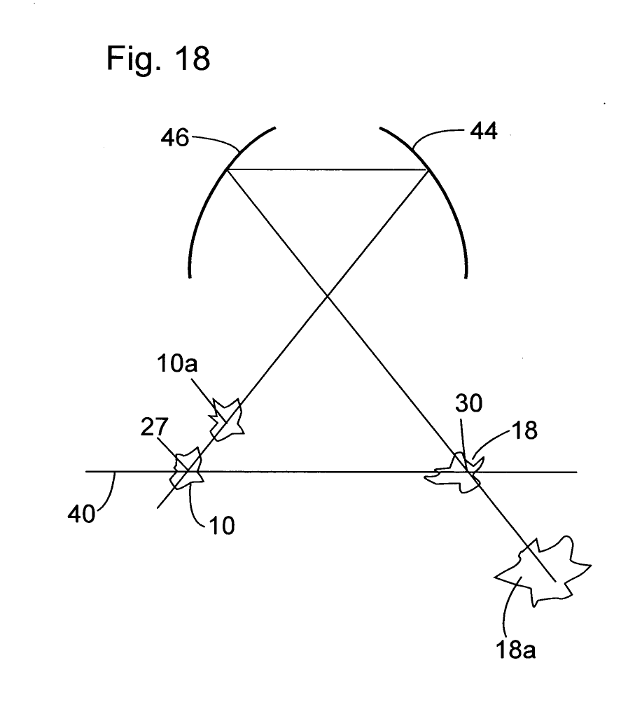

In the embodiments disclosed in FIGS. 5 and 7, the object 10 is placed at the focus of the first paraboloid reflector. As a result, the image 18 has the same size as the object, i.e., there is unit magnification. In FIG. 18, a first object 10 and image 18 have the same configuration, i.e., object 10 and image 18 are at the focal points 27, 30 of the dual paraboloid reflectors 44, 46. In FIG. 18, a second object 10a is placed away from the focal point 27 in a direction closer to the reflector 44. As a result, a magnified image 18a is formed at a location away from the focal point 30 further from the second paraboloid reflector 46. The amount of magnification, and thus the size of the image 18a, depends upon the distance by which the object 10a is moved away from the focus 27. Note also that, if the object 10a is contained inside a kiosk, the image 18a can be formed outside of the kiosk.

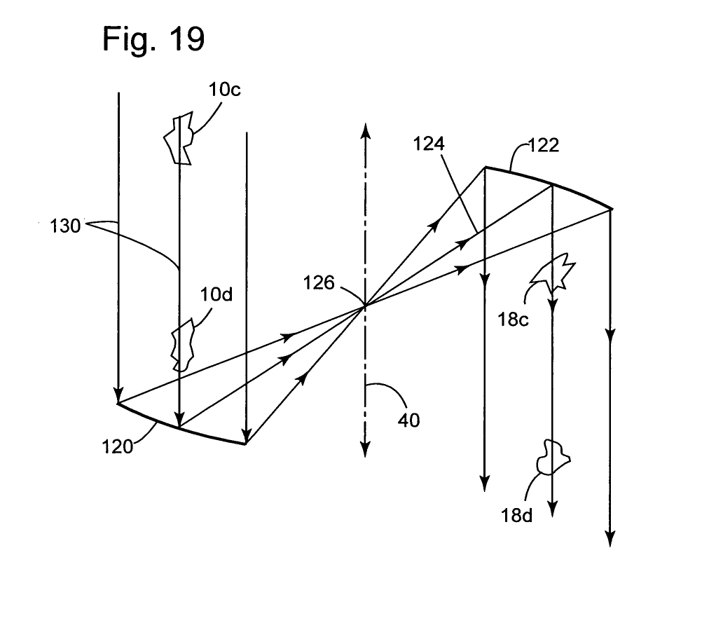

FIG. 19 schematically shows another embodiment of a dual paraboloid reflector system in which the two reflectors 120, 122 have a common central axis 124 (are pointed towards one another) and are spaced relative to one another such that they share a common focus 126. Both reflectors thus share a common axis of rotation 40 (in this disclosure, axis of rotation and axis of revolution, in connection with a parabolic surface, are used interchangeably). The object 10 is placed in front of the reflector 120 at a sufficient distance (optical infinity) such that beams 130 which represent the object are transmitted to the first reflector 120 along essentially a collimated path 130. Collimated beams of light 130 which impact the first reflector 120 are reflected through focus 126 and imaged as an inverted image on the second reflector 122, which in turn creates a collimated output image.

In using the system shown in FIG. 19, an object 10c placed at a first location will produce a floating image 18c. In the case of object 10c, the image 18c will have unit magnification. An object 10d which is located closer to the first reflector 120 will produce a floating image 18d which is further away from the second paraboloid reflector 122, but retain unit magnification. This embodiment has the advantage that by moving the object, the resulting floating image 18c, 18d will appear to be moving in space. The image 18c, 18d will also appear to move side-to-side when the object 10c is moved perpendicular to the parallel beams 130.

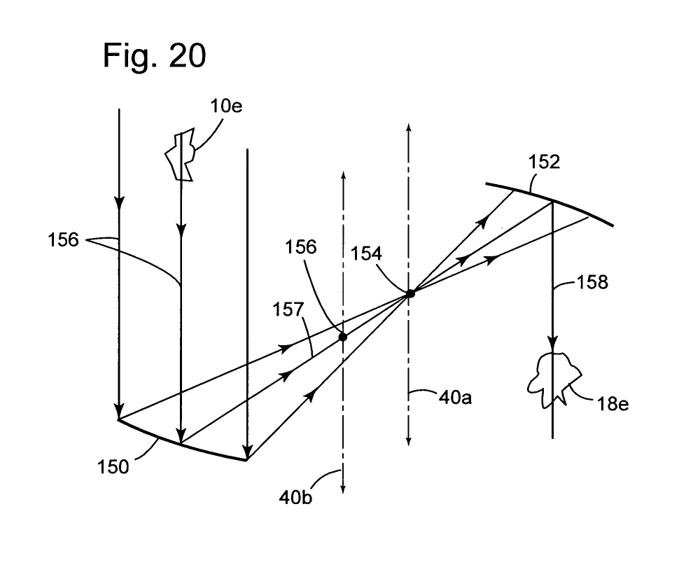

FIG. 20 schematically shows another embodiment of the invention in which the distance between the paraboloid reflectors 150 and 152 is shortened so that the focus 154 of the first paraboloid reflector 150 and the focus 156 of the second paraboloid reflector 152 are not coincident. In particular, the focus 154 is closer to the second paraboloid reflector 152 and vice versa. The axis of rotation 40a of the first reflector 150 is likewise closer to the second reflector 152, and the axis of rotation 40b of the second reflector 152 is closer to the first reflector 150 than to the second reflector 152. As a result, when an object 10e is placed at optical infinity relative to the reflector 150, light rays are transmitted to the reflector 150 as collimated light 156, and the image 18e formed is larger than the object 10e. However, similar to FIG. 19, when the object 10e is moved along the input beam path 156, the output floating image 18e also moves along the output beam path 158 without changing size.

The above descriptions of FIGS. 19 and 20 apply for off-axis paraboloid reflectors with angles of 45 degrees (meaning that the object 10 is placed along an axis which intersects the first reflector and which axis lies at an angle of 45 degrees relative to the optical path between the reflectors). The same principle can be applied to off-axis paraboloid reflectors with other off-axis angles, e.g., 30, 60, 90 degrees, etc.

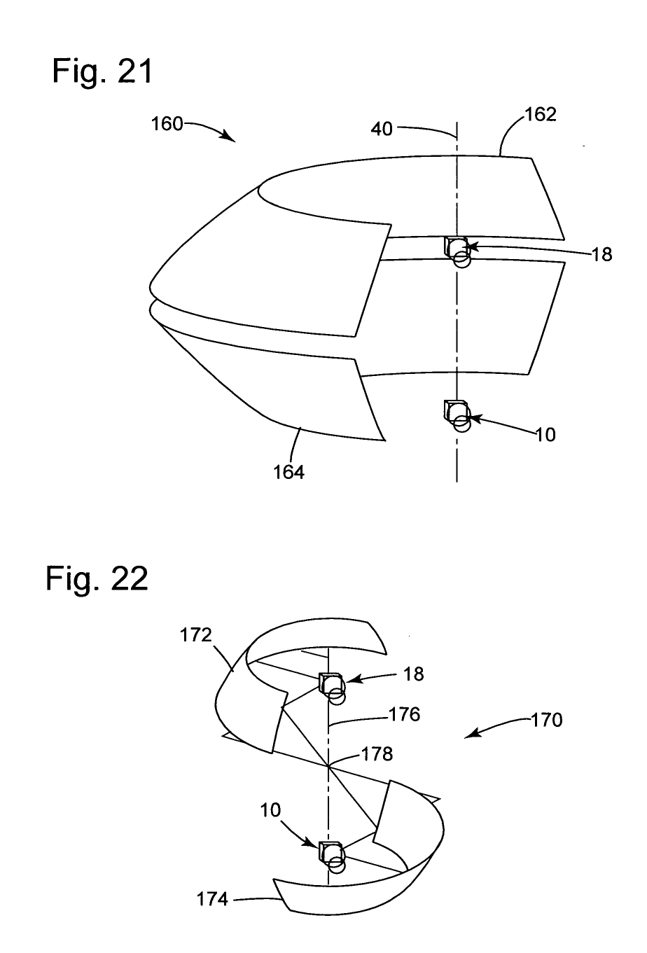

FIG. 21 schematically shows in three dimensions a dual paraboloid reflector panoramic 3-D floating image system 160. The system 160 includes two 90 degree off-axis paraboloid reflectors 162, 164 sharing a common axis of rotation 40 and facing one another symmetrically. As shown, an object 10 is placed along the axis of rotation so that it is at the focal point of the first paraboloid reflector 164. The reflectors 162, 164 then form a floating image 18 at a focal point of the second paraboloid reflector 162. Since the lines between the image transmission path between the object 10 and the image 18 on each half of the system do not cross one another, the reflectors 162, 164 may subtend an angle around the axis of rotation 40 up to a full revolution of 360 degrees for a full immersive experience. As shown in the figure, there is 180 degrees of revolution in the horizontal plane—theoretically, there could be up to 360 degrees of revolution, completing a circular reflector. For table tops and store windows, an angle up to 180 degrees is a practical limit to the system. Since the viewer is not inside the reflectors, one has to view one-half of the circle from the other half.

FIG. 22 shows a dual elliptical reflector panoramic 3-D floating image system 170. The system includes two elliptical reflector sections 172, 174 whose reflective surfaces face one another. The two reflector sections 172, 174 share the same axis of rotation 176 and have a common focus point 178. Similar to the dual paraboloid reflector systems described herein, the full angle can be 360 degrees with the practical extent in common systems being 180 degrees. The common focus with a smaller beam size allows easier rerouting of the light path in order to fit particular constraints of the system. Parallel beams have large physical extent compared to focused beams.

The two reflector sections 172, 174 may be the same size, or may be different sizes as long as the ellipticities of the two reflectors are the same. As a result, smaller or larger reflectors can be used depending on the mechanical constraints of the system.

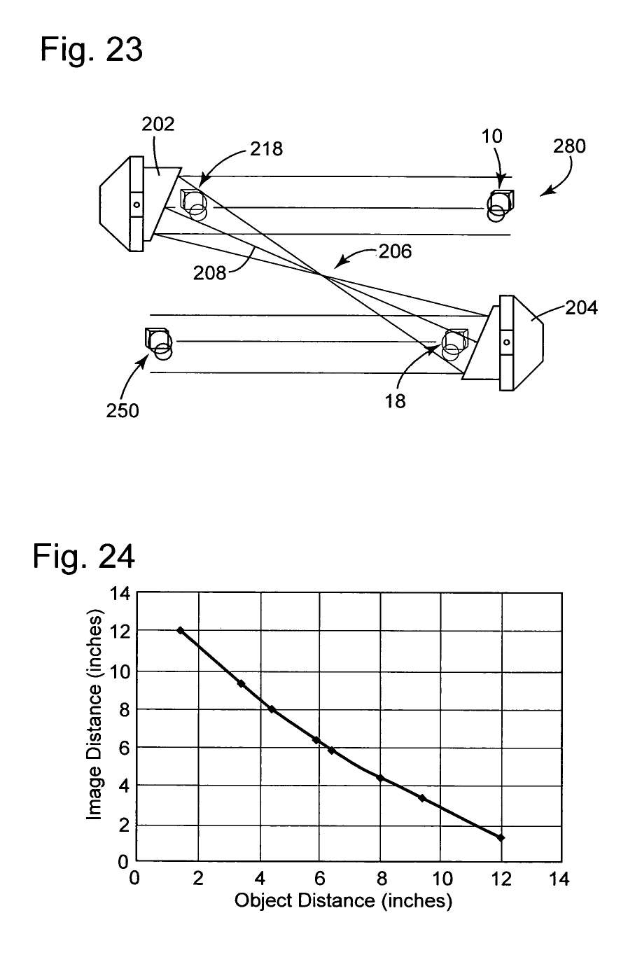

FIG. 23 shows a four-dimensional floating image display system 280. The system includes two off-axis paraboloid reflectors 202, 204 configured to have a common focus 206 and co-linear optical axes 208. An object 10 is placed at optical infinity in front of the first reflector out the path of the optical axis 208 (thus, off-axis), such that the light rays representing the object's image are collimated when they reach the first reflector 202. The light rays reflected from the first reflector are directed to the second reflector 204 along optical path 208, where they are further reflected forming a real 3-D image 18 in front of the second reflector 204. Because the system is symmetric, when an object 250 is placed in front of the second reflector 204, a real 3-D image 218 will be formed in front of reflector 202. The property of the fourth dimension is realized by moving the object 10 or 250 toward the respective reflector 202 or 204. In each case, moving the object towards the reflector will cause the image to move away from the other reflector without changing size.

A prototype of the system 200 was built using two 75 mm, 45-degree off-axis paraboloid reflectors with a focal length of 150 mm. The two reflectors were positioned to have a common focal point 206, as shown in FIG. 23. The object 10 was moved relative to the first reflector 202 and its distance from the first reflector, as well as the distance of the image 18 from the second reflector, were measured. The results, shown in FIG. 24, indicate that the image movement substantially corresponded to the movement of the object.

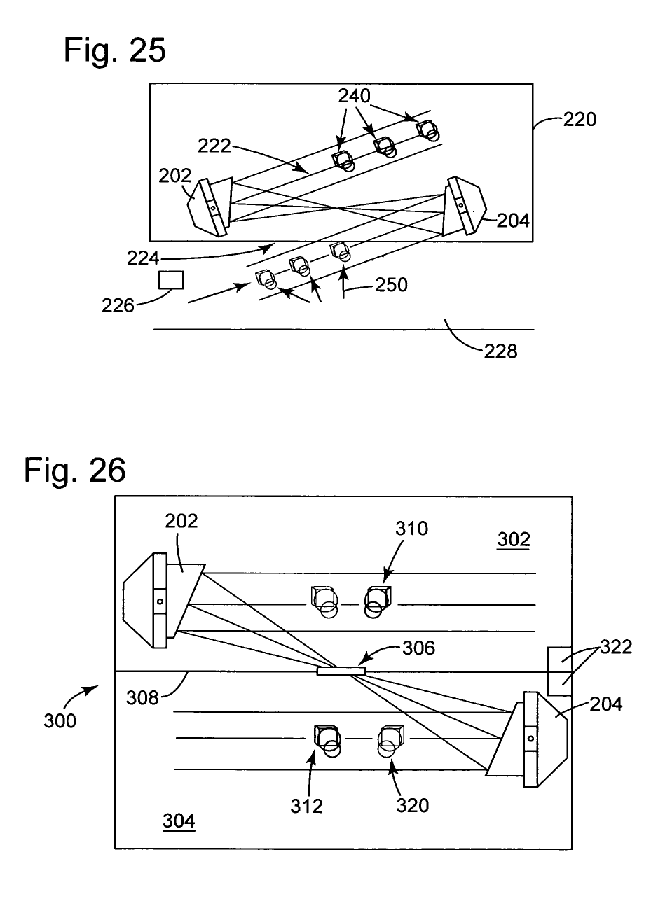

FIG. 25 shows an application of the 4-D system 200 shown in FIG. 23. The dual paraboloid reflectors 202, 204 are placed inside a building such as a department store 220 having a front window 224. A runway 222 is arranged to be off-axis with respect to the first paraboloid reflector 202, and the reflectors are oriented and spaced from one another as described in FIG. 23. When an object, such as a fashion model 240, walks down the runway 222, the model's image is projected out the front window 224 and appears to pedestrians 226 on the sidewalk 228 as a 3-D floating, moving image 250.

FIG. 26 schematically illustrates another application of the system of FIG. 23. In the system 300 shown in FIG. 26, the two paraboloid reflectors 202, 204 are placed in two separate rooms 302, 304. A glass window 306 is provided in the wall 308 separating the two rooms. The two reflectors are oriented and spaced from one another so that the common focus is located in the window 306. In use, a first person 310 appears before the first reflector 202 in the first room 302, and another person 312 appears before the second paraboloid reflector 204 in the second room 304. As shown, a 3D image of the first person 310 appears between the second person 312 and the second reflector 204. Similarly, a 3-D image of the second person 312 appears between the first person 310 and the first reflector 202. By setting up and connecting audio equipment 322 in each room the first and second persons 310, 312 may communicate with one another, both visually and audibly, without being physically present in the same room. The system 300 will be especially useful where, for security reasons, it is desirable to keep two people separated from one another, such as an attorney from an incarcerated inmate, or a bank teller from a bank customer.

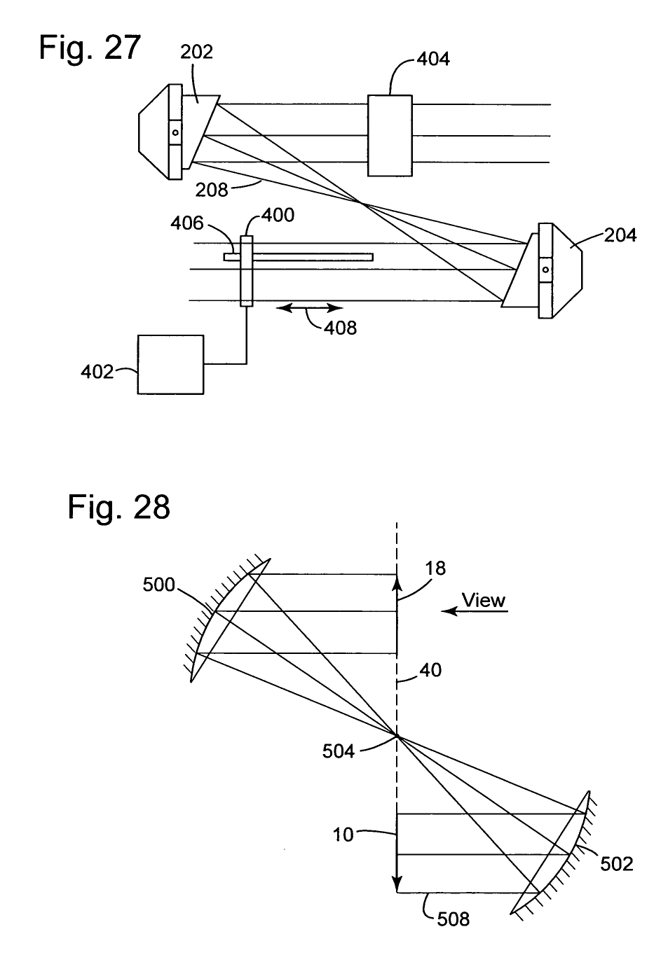

FIG. 27 illustrates another application of the display system of FIG. 23. The object used in FIG. 27 constitutes an LCD panel 400 which is controlled by a computer 402 to project and display 3D images 404. Information used to create a 3-dimensional image is referred to as “voxels.” Voxels are the 3-D equivalent of pixels used in a 2-D display. In essence, the image to be displayed is represented by a plurality of many slices. The voxels for displaying each slice are processed by the computer 402 such that the voxels for each layer of the image to be displayed sequentially by the LCD panel 400 for a predetermined period of time. The combination of these layers thereby create a 3-D image.

In the case of FIG. 27, the LCD panel 406 is positioned at a predetermined location and a slice of the image 404 is displayed at location 404. The LCD panel 406 is moved slightly to a second predetermined position, and the computer supplies a second set of voxels representing the second slice of the image. The second slice is projected by the LCD panel 406 and displayed at the location 404. This process is repeated until the full 3-dimensional image 404 has been displayed. Movement of the LCD panel 406 is sufficiently quick that the image 404 appears to the viewer to be a solid image.

Once a full image 404 has been displayed, the process repeats for form additional images. Each subsequent image may change from the prior image, for example to show a person performing an action such as walking, talking, or displaying an object. Moreover, by moving the location of the LCD panel 406, the image 404 actually moves in space. Accordingly, an image showing a person walking can be made to move by the same amount and appear actually to be walking. Thus, by way of example, the LCD panel 400 can form a 3-dimensional image 404 of a model actually walking down a runway.

For a simple translation of the LCD panel 400, the panel 400 can be mounted on a track 406 and moved by a linear motor (not shown) parallel to the beam path 408. Motor rotation is preferably controlled by the computer 402. The LCD panel 400 is moved quickly the short distances needed to create each subsequent slice of the instantaneous image 404, and can be additionally moved linearly at a slower rate to cause the image to move relative to the reflectors.

Alternately, the panel 400 can be mounted on a turntable 406a. In the latter case, as the turntable 406a rotates, the LCD panel 400 will also move perpendicular to the beam path 408. The displayed images can be modified accordingly by the software creating the displayed 3-D images

In known floating image systems, the real image floating in the air usually has a narrow angle of view. Also, the image tends to be distorted depending on the off-axis viewing angle. FIGS. 28, 30-33, and 35 disclose floating image systems, using at least two paraboloid reflectors, in which the real image formed by the system can be viewed at a large off-axis angle without distortion. The extent of the viewing angle from which the image can be viewed without distortion is determined by the width of the reflectors.

FIG. 28 shows a first embodiment of a wide angle floating image system. A pair of paraboloid reflectors 500, 502 share a common axis of rotation 40 and a common focal point 504. The focal point 504 lies in the axis of rotation 40. The reflectors 500, 502 are positioned to have the same off-axis angle on opposite sides of the axis of rotation 40 so as to face one another as shown. The object 10 is placed in the axis of rotation 40, such that the system will create an image 18 of the object 10 which is also in the axis of rotation 40 from which the paraboloid reflectors 500, 502 are formed.

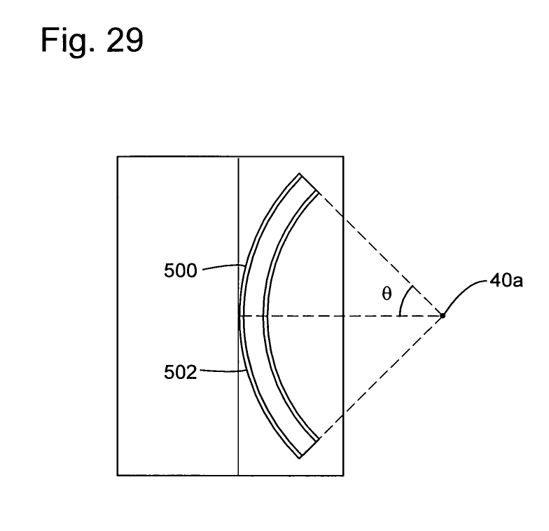

Collimated light beams 508 from the object 10 are focused by the paraboloid reflector 502 at focal point 504 and sent to the other paraboloid reflector 500, which collimates the beams 508 to form the image 18 at the axis of rotation 18. The extent of the reflectors 500, 502 around the axis of rotation 40 is shown in FIG. 29 with a half angle θ. With such configuration and angular symmetry around the axis of rotation, the real image 18 formed from the object 10 at the axis of rotation 40 will be viewable around the axis of rotation 40 within a field of viewing angle of plus/minus θ with minimal distortion.

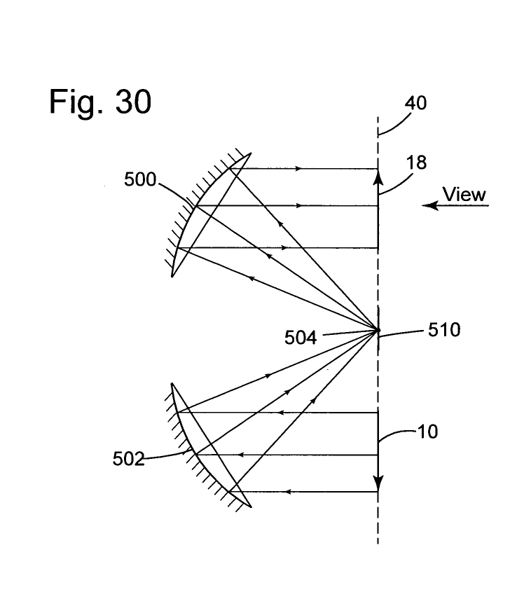

In order to reduce the overall size of the system, the reflectors 502 can be folded back on itself to lie on the same side of the axis of rotation 40 as the reflector 500, as shown in FIG. 30. In the FIG. 30 alternative embodiment, a flat mirror 510 is placed at the common focal point 504 to reflect beams from the reflector 502 towards the other reflector 500.

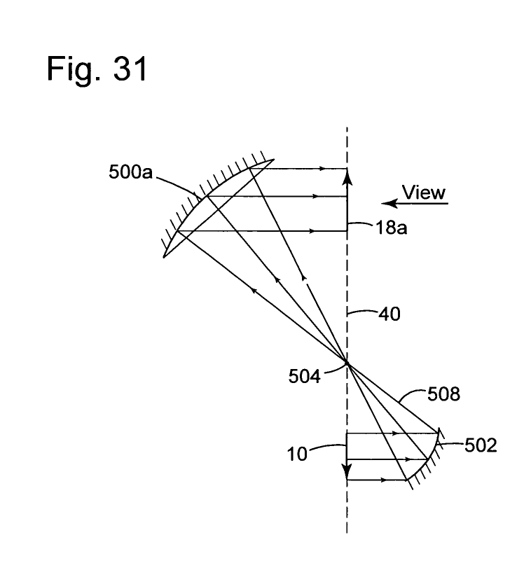

The embodiments disclosed in FIGS. 28 and 30 use the same size parabolic reflectors 500, 502 at the same distance from the axis of rotation in a symmetrical arrangement. Thus, the image 18 formed is the same size as the object 10. FIG. 31 shows a variation of such configurations to produce a magnified image 18a. To do so, one parabolic reflector 500a has a longer focal length than the parabolic reflector 502 which first receives the beams of light from the object 10. The amount of magnification produced depends on the ratio of the focal lengths of the two reflectors 500a, 502. Again, the object, image, and shared focal point are on the same axis of rotation 40 from which the parabolic reflectors are formed. And, in order to form a complete image 18a, the paraboloid reflector 500a must have a size large enough to receive all of the light beams 508 reflected by the first paraboloid reflector 502.

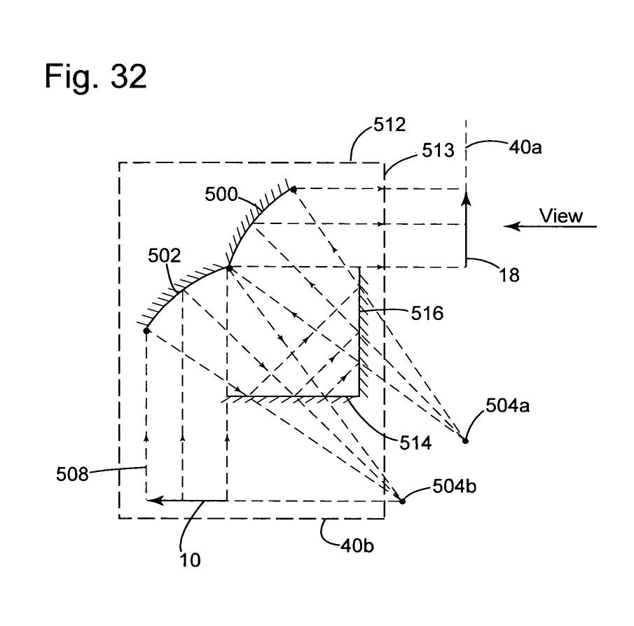

FIG. 32 shows another alternative embodiment of a wide angle floating image system which is contained in a housing 512 (which may be a kiosk). One paraboloid reflector 500 has an axis of rotation 40a which is located in front of the housing 512. The other paraboloid reflector 502 has an axis of rotation 40b which is located inside the housing 512. The axis of rotation 40b is perpendicular to the axis of rotation 40a and intercepts axis 40a. A pair of flat mirrors 514, 516 are at 90 degrees relative to one another, with mirror 514 being parallel to the axis of rotation 40b and the mirror 516 being parallel to the axis of rotation 40a. One mirror is located between each reflector 500, 502 and its corresponding focal point.

The object 10 is placed inside the housing 512 on the axis of rotation 40b of the reflector 502. Parallel beams 508 representing the shape of an object 10 are received by the reflector 502 and partially focused onto the flat mirror 514, which in turn reflects the partially focused beams onto the second mirror 516, which directs the beams to the other reflector 500. The beams received by the other reflector 500 are collimated and appear as image 18 at the axis of rotation 40a. Due to the double reflection by flat mirrors 514, 516 light beams travelling between the mirror 516 and the other reflector 500 expand in the same manner as light beams contract when travelling between the reflector 502 and the flat mirror 514. As a result, the image received on the surface of the reflector 502 will be recreated on reflector 500. The beams are collimated by the other reflector 500 and directed through a hole 513 in the housing 512 to the axis of rotation 40a.

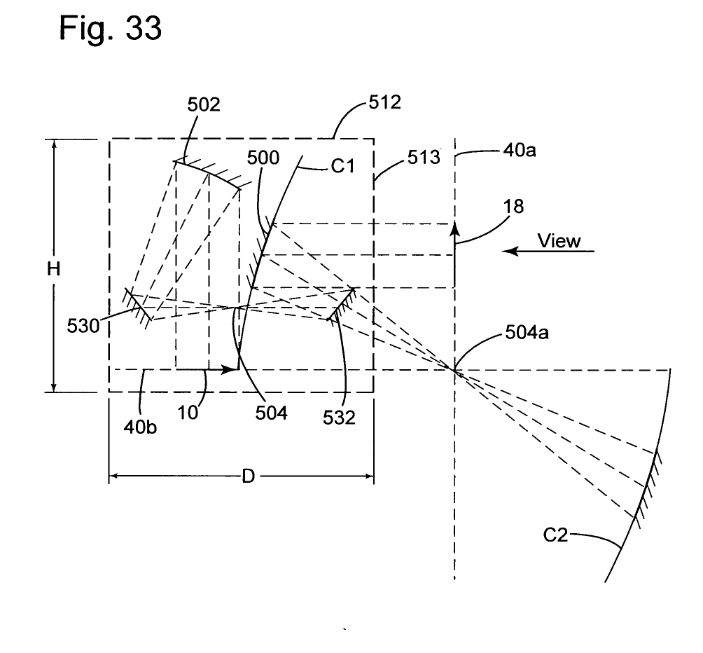

FIG. 33 shows another alternative embodiment of a wide angle floating image system. As in the case of FIG. 32, the paraboloid reflector 500 is formed along an axis of rotation 40a which is perpendicular to, and which intercepts, the axis of rotation 40b of the reflector 502. The axis of rotation 40a, on which the image 18 will be formed, is located in front of the housing 512.

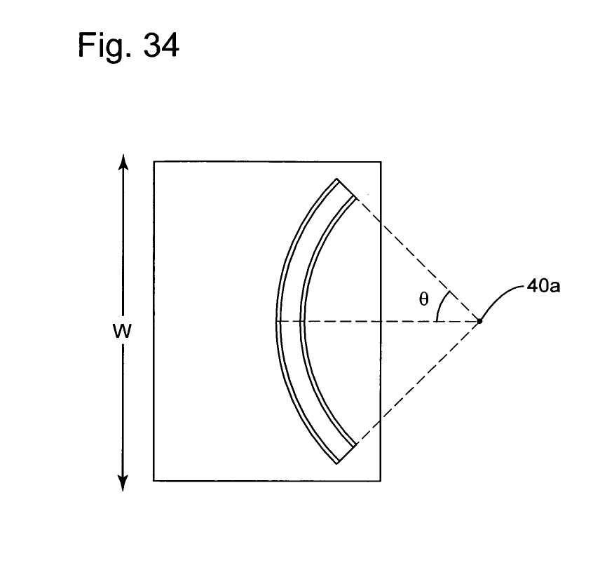

Collimated beams of light representing the shape of the object 10 are received on the paraboloid reflector 502 and partially focused on a mirror 530. The mirror 530 directs the beams onto a second mirror 532, which reflects the beams onto the other paraboloid reflector 500. The other reflector 500 collimates the beams, and forms an image 18 at its axis of rotation 40a. The width of the system is determined by the viewing angle as shown in FIG. 34.

FIGS. 32 and 33 show examples of how the light paths from the object to the image can be folded to achieve designs with maximum floating distance in front of the housing 512 with minimum housing size (dimensions). Other possibilities and designs using similar principles will be evident to persons skilled in the art.

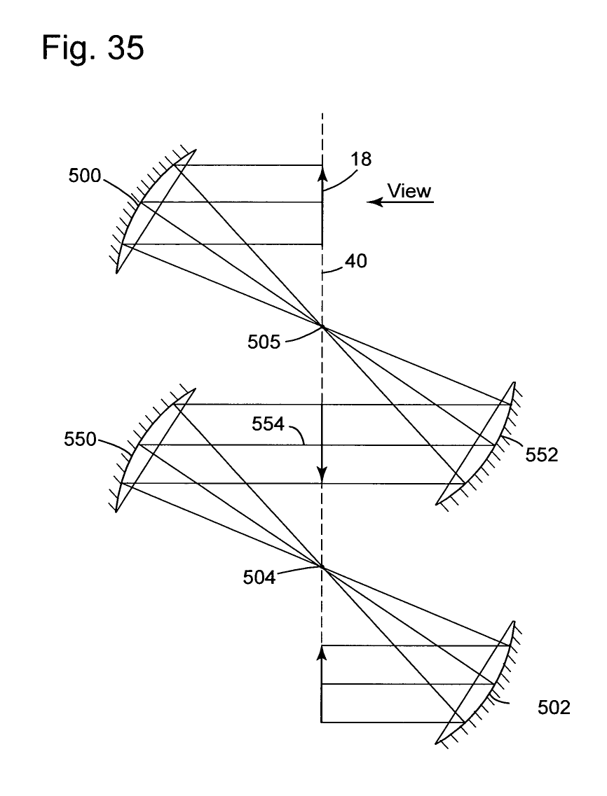

FIG. 28 shows a basic configuration in which the real floating image 18 is inverted relative to the object 10. In order to produce an upright real floating image, and additional stage may be added, as shown in FIG. 35. The inverted intermediary image of the object 10 from reflector 550 is inverted by reflectors 552 and 500 to produce a final image 18. Similar to previous embodiments, the first reflector pair 502, 550 share a common axis of rotation 40 and focal point 504. The second reflector pair 552, 500 also share a common axis of rotation 40 and focal point 505. And, all four reflectors 500, 502, 550, and 552 share the same axis of rotation 40. Each reflector of each pair is located and oriented to receive the inverted image from the other reflector of the pair. And the reflector 552 is located and oriented to receive the collimated beams 554 from the second reflector 550 of the first pair of reflectors 502, 550.

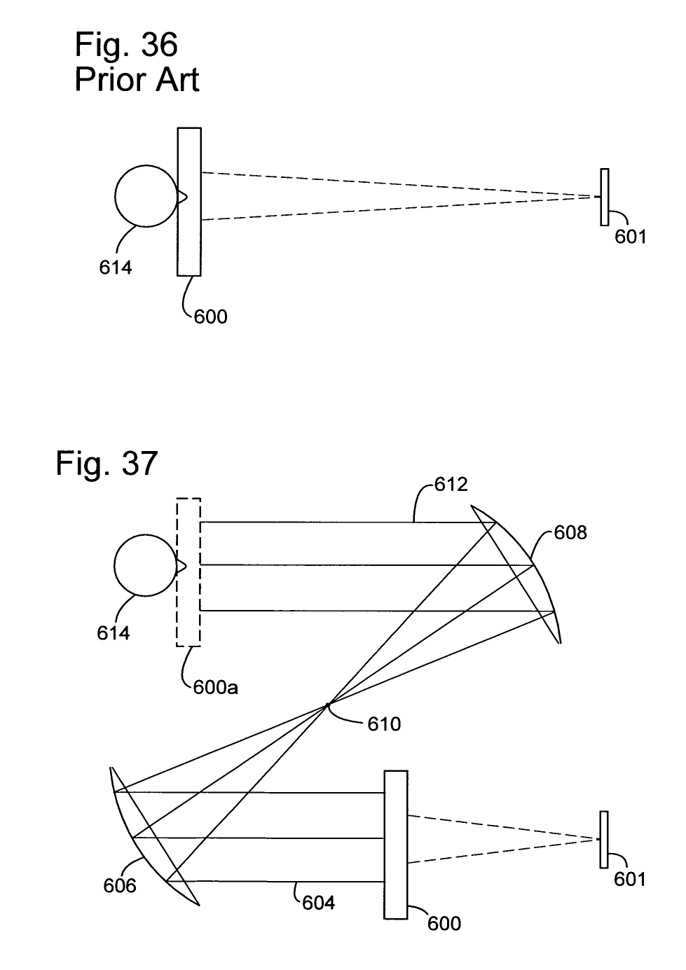

FIG. 36 is shows a typical arrangement for determining an eyeglass prescription for a patient needing glasses. The patient sits behind a phoroptor 600, which is an instrument containing different lenses used for refraction of the eye during sight testing. For prescribing distance lenses (myopia), the patient looks through the phoroptor, one eye at a time, at an eye chart 601 placed at optical infinity (approximately 6 meters). The patient then tries to read different lines on the eye chart which vary in size. During the process, the eye care professional changes lens and other settings on the machine, using various dials, while asking the patient for subjective feedback on which settings gave the clearest image. The final prescription is determined when the patient can read the eye chart clearly. After the prescription is determined for one eye, the process is repeated for the other eye. The phoroptor 600 may also be used to determine a prescription for reading glasses.

The phoroptor 600 is a large, bulky piece of equipment that needs to be placed in contact with the patient's face. The phoroptor machine 600 must be designed to provide an opening between two sections of the machine in order to accommodate the patient's nose. Older machines are operated manually, with the eye care professional standing next to the machine. Because the phoroptor is made movable using articulated arms, the machine tends to be limited in functions. Newer, automated phoroptors are controlled by remote terminals and usually designed with the same overall size as the manual machines. Like the older machines, the newer machines require that the patient's face be in physical contact with the machine in order to precisely position the patient's eyes relative to the lens and to accommodate the patient's nose. This again limits the functioning of the machine, and the limitations in weight and size have prevented the development of machines which are more sophisticated and comfortable for the patient.

FIG. 37 shows an embodiment of a 4-D system for determining an eyeglass prescription for a patient needing reading glasses, distance glasses, or bifocal lenses. The system can employ any of the previously discloses systems, e.g., in FIG. 19, 20, 23, 25-28, 30-33, or 35, in which a pair of reflectors are positioned to reflect collimated beams representing an object at a first location, as a second set of collimated beams forming an image along a second path. Collimated beams 604 representing an actual phoroptor 600 at a first location are reflected by a first reflector 606 to a second reflector 608. The two reflectors 606, 608 share a common focal point 610 such that the beams 612 emitted by the second reflector 608 are collimated.

When conducting an eye examination, the patient 614 is positioned in the path of the reflected beams 612 such that the patient sees a precise image 600a of the phoroptor 600. The machine 600 can be adjusted and repositioned such that the real image 600a will be at the correct position with the patient 614 looking through the real image 600a as if his or her face were positioned in contact with the actual phoroptor 600. Because the patient is looking through the changeable lenses of the phoroptor 600 as if he or she was actually sitting in front of the machine 600, the patient can view any suitable eye chart 601 to select the lens which provides the clearest image.

With the present invention, the patient will be more comfortable insofar as there is no need to be physically in contact with the machine 600. Elimination of the need for physical contact between the machine 600 and patient 614 eliminates any risk of transmission of disease from the machine 600 to the patient 614, or from one patient 614 and the next patient. The eye care professional can then determine the proper prescription using the actual phoroptor machine 600 while standing at a distance away from the patient for a manual machine, and at the terminal for automatic machines 600, the same way as if the patient were physically present at the machine 600.

Because the phoroptor machine 600 is remote from the patient, there are no limitations as to its weight, dimensions, or shape. Thus, it is no longer necessary to design the housing with a cutout portion for the patient's nose. It also is possible, instead of using articulation, to move and adjust the machine's position with any suitable mechanism, such as a heavy-duty mover, while the patient remains stationary.

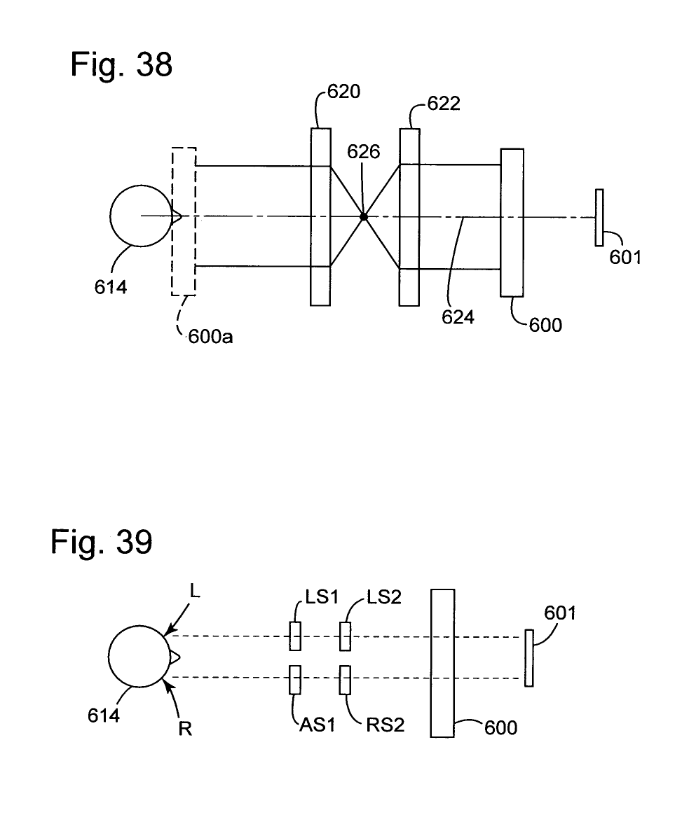

FIG. 38 shows an alternative embodiment of a system for conducting eye examinations. A pair of lenses 620, 622 are aligned along common optical axis 624 and spaced from one another to share a common focal point 626. The phoroptor machine 600 is placed in front of one lens 622, and the patient is placed on the opposite side of the two lenses 620, 622.

In certain optical applications, using lenses provides a higher optical precision than using reflectors. For such applications, relocation of the phoroptor machine 600 can be done using high precision lens systems such as shown in FIG. 39. The system shown in FIG. 39 is divided into two sections, separating the left eye optics from the right eye optics. More particularly, the optics for the left eye of the patient 614 include a pair of lenses LS1 and LS2. The optics for the right eye include lenses RS1 and RS2. The lenses LS1 and LS2, and RS1 and RS2, are positioned as shown in FIG. 38, to share a common focal point 626L and 626R. The machine 600 is positioned so that the patient's left eye L is in the path of the lens pair LS1, LS2, and the patient's right eye R is in the path of the lens pair RS1, RS2.

The paraboloid, ellipsoid, and spherical reflectors can be made from metal, plastic, glass, or any other suitable material. They can be machined or molded using high precision molds. The reflectors can be coated with metal or a multi-layer dichroic coating.

The object can be an actual product such as shoes, diamond rings, toys, etc. and the dimension of the kiosk and reflectors can scaled based on the desired size of the projected image.

The object can also be a fixed or digital display screen, e.g., LCD, plasma, OLED, and so on. The projected image will then be that of the display screen.

The object can also be a combination of one or more real physical objects together with a display screen such that the object can be viewed with various backgrounds or graphics.

The foregoing represent preferred embodiments of the invention. Variations and modifications of the embodiments will be evident to persons skilled in the art. All such variations and modifications are intended to be part of the invention, as defined by the following claims.

1. An aerial projection display for projecting an image from an object, located at a first location, to a second location, said display comprising:
a pair of spherical, parabolic, or elliptical reflectors positioned and oriented relative to one another such that, when the object is placed at one or more predetermined locations, an image of such object is transmitted to a first reflector of said pair, reflected by said first reflector to a second reflector of said pair, and projected by said second reflector as an undistorted 3-D image of the object at the second location.

2. The display of claim 1, wherein said first and second reflectors are paraboloid reflectors sharing a common axis of revolution and having portions which meet along a common plane, wherein said reflectors have first and second focal points, respectively.

3. The display of claim 2, wherein said predetermined location is said first focal point.

4. The display of claim 3, wherein said axis of revolution is spaced away from said reflectors.

5. The display of claim 2, wherein said predetermined location is spaced away from said first focal point.

6. The display of claim 2, wherein said reflectors face one another and have a common central axis 124, wherein predetermined location is at an angle relative to such axis, and wherein collimated light from said predetermined location which impacts said first reflector is reflected toward said second reflector, and then reflected by said second reflector as an image of collimated light in a direction parallel to the collimated light from the object, wherein moving the object causes the image to move by a comparable amount.

7. The display of claim 6, wherein said reflectors share a common focus.

8. The display of claim 1, wherein said reflectors are elliptical reflectors each having a pair of foci, wherein said foci lie along a common axis, and wherein one of said foci is common to both reflectors.

9. The display of claim 8, wherein one of said reflectors is larger than the other for magnifying the image of the object.

10. The display of claim 1, wherein said reflectors are spherical reflectors, wherein the first reflector reflects a distorted image of the object to said second reflector, and wherein said first and second reflectors are oriented relative to said predetermined location such that the second reflector corrects the distorted image from said first reflector to project a substantially undistorted image of the object.

11. The display of claim 2, further comprising a kiosk having an interior for containing the object and the reflectors.

12. The display of claim 8, further comprising a kiosk having an interior for containing the object and the reflectors.

13. The display of claim 10, further comprising a kiosk having an interior for containing the object and the reflectors.

14. The display of claim 2, further comprising a building having a window, wherein said second reflector is positioned to project an object inside the building through the window and refocus the object outside of the building.

15. The display of claim 2, further comprising a structure having two rooms separated by a wall, wherein said wall includes a window, wherein said reflectors are located in separate rooms, and wherein said first reflector is oriented to reflect the image of the object through the window to the second reflector, for creating an image of the object in the second room.

16. The display of claim 15, wherein the image moves commensurate with movement of the object to create a virtual presence of the object in the second room.

17. The display of claim 16, further comprising an audio system for transmitting sound between the two rooms.

18. The display of claim 1, wherein the two reflectors are parabolic reflectors having a common focal point, wherein said predetermined location is off-axis relative to one of the reflectors and at optical infinity such that light rays from said object which reach the one reflector are collimated; wherein the one reflector focuses the rays at the common focal point, and wherein the other reflector projects the beams as collimated to form an image.

19. The display of claim 18, wherein the object is an image display for projecting a moving image.

20. The display of claim 19, wherein the image display creates a 3-D image.

21. The display of claim 20, wherein the image display is moveable towards and away from the one reflector and produces a 3-D image by projecting sequential slices of the object while being moved toward and away from the one reflector.

21. The display of claim 21, wherein the image display is further moveable to cause the projected image to move.

22. A method for determining an eyeglass prescription for a patient comprising the display of claim 18, wherein the object is a phoroptor for displaying plurality of lenses and an eye chart visible to the patient through each of the lenses; the method comprising the steps of locating the phoroptor at a predetermined location so as to project an image of the phoroptor at a second location, and displaying a plurality of lenses to the patient while the patient is positioned to view the projected image.
